(19) United States
(12) Patent Application Publication
(10) Pub. No.: US 2022/0313214 A1
(43) Pub. Date: Oct. 6, 2022

(54) ULTRASONIC DIAGNOSTIC APPARATUS, IMAGE PROCESSING APPARATUS, AND IMAGE PROCESSING METHOD

(71) Applicant: CANON MEDICAL SYSTEMS CORPORATION, Otawara-shi (JP)

(72) Inventors: Koji ANDO, Otawara (JP); Mitsuo AKIYAMA, Otawara (JP); Kodai HIRAYAMA, Nasushiobara (JP); Yasuhiko ABE, Otawara (JP); Koichiro KURITA, Nasushiobara (JP)

(73) Assignee: CANON MEDICAL SYSTEMS CORPORATION, Otawara-shi (JP)

(21) Appl. No.: 17/657,153

(22) Filed: Mar. 30, 2022

(30) Foreign Application Priority Data
Apr. 2, 2021 (JP) 2021-063810

(51) Int. Cl. A61B 8/00; A61B 8/08

(52) U.S. Cl. CPC A61B 8/463; A61B 8/0883

(57) ABSTRACT

The ultrasonic diagnostic apparatus according to any of embodiments includes processing circuitry. The processing circuitry is configured to acquire ultrasonic image data by ultrasonic scanning. The processing circuitry is configured to acquire multiple contour candidates based on a quantitative value indicating a function of a tissue, and superimpose the multiple contour candidates on the acquired ultrasonic image data to display the multiple contour candidates on a display. The processing circuitry is configured to determine contour data out of the multiple contour candidates.

### CROSS-REFERENCE TO RELATED APPLICATION

This application is based upon and claims the benefit of priority from Japanese Patent Application No. 2021-063810, filed on Apr. 2, 2021, the entire contents of which are incorporated herein by reference.

### FIELD

Any of embodiments disclosed in specification and drawings relates to an ultrasonic diagnostic apparatus, an image processing apparatus, and an image processing method.

### BACKGROUND

In the medical field, an ultrasonic diagnostic apparatus is used for imaging the inside of a subject using ultrasonic waves generated by multiple transducers (piezoelectric vibrators) of an ultrasonic probe. The ultrasonic diagnostic apparatus causes the ultrasonic probe, which is connected to the ultrasonic diagnostic apparatus, to transmit ultrasonic waves into the subject, generates an echo signal based on a reflected wave, and acquires a desired ultrasonic image based on the echo signal by image processing.

In echocardiography using the ultrasonic diagnostic apparatus, the operator manually selects the optimal heartbeat out of the most recent multiple heartbeats after the display image freeze operation, and then performs various measurements and analyzes on the ultrasonic image corresponding to the selected heartbeat. The method for measuring and analyzing the ultrasonic image may include two-dimensional or three-dimensional WMT (Wall Motion Tracking) for analyzing the wall motion of the myocardium, and “Auto EF” (Automated Ejection Fraction) that automatically calculates the left ventricular ejection fraction and the like.

The “Auto EF” is a method of performing pattern recognition by comparing the actual morphology of the heart with the characteristics registered in the pre-constructed database (appearance of the heart, left ventricular endocardium, etc.) to search for a heart with a similar pattern so as to detect the endocardium of the left-ventricular and to calculate the end-diastolic volume (EDV), the end-systolic volume (ESV), the ejection fraction (EF), or the like as a quantitative value indicating the cardiac function in each heartbeat.

### BRIEF DESCRIPTION OF THE DRAWINGS

FIG. 1 is a schematic view showing an example of a configuration of the ultrasonic diagnostic apparatus provided with the image processing apparatus according to the first embodiment.

FIG. 2 is a block diagram showing an example of functions of the image processing apparatus according to the first embodiment.

FIG. 3 is a diagram showing a display example of a B-mode image in the image processing apparatus according to the first embodiment.

FIG. 4 is a diagram showing a display example of a thumbnail image in the image processing apparatus according to the first embodiment.

FIG. 5 is a flowchart showing an example of an operation of the image processing apparatus according to the first embodiment.

FIG. 6 is an explanatory diagram showing an example of a data flow during learning of the image processing apparatus according to the first embodiment.

FIG. 7 is an explanatory diagram showing an example of a data flow during operation of the image processing apparatus according to the first embodiment.

FIG. 8A shows the first display screen including contour candidates in the image processing apparatus according to the first embodiment.

FIG. 8B shows the second display screen including contour candidates in the image processing apparatus according to the first embodiment.

FIG. 9 shows the third display screen including contour candidates in the image processing apparatus according to the first embodiment.

FIG. 10 is a schematic view showing an example of a configuration of a medical image system including the image processing apparatus according to the second embodiment.

FIG. 11 is a block diagram showing an example of functions of the image processing apparatus according to the second embodiment.

FIG. 12 is a flowchart showing an example of an operation of the image processing apparatus according to the second embodiment.

### DETAILED DESCRIPTION

An ultrasonic diagnostic apparatus, an image processing apparatus, and an image processing method according to any of embodiments will be described with reference to the accompanying drawings.

The ultrasonic diagnostic apparatus according to any of embodiments includes processing circuitry. The processing circuitry is configured to acquire ultrasonic image data by ultrasonic scanning. The processing circuitry is configured to acquire multiple contour candidates based on a quantitative value indicating a function of a tissue, and superimpose the multiple contour candidates on the acquired ultrasonic image data to display the multiple contour candidates on a display. The processing circuitry is configured to determine contour data out of the multiple contour candidates.

The image processing apparatus according to any of embodiments is provided as a part of a medical image diagnostic apparatus that generates a medical image. Hereinafter, in the first embodiment, a case where the image processing apparatus is provided as the medical image diagnostic apparatus of the ultrasonic diagnostic apparatus will be described. However, it is not limited to that case. For example, the image processing apparatus may be provided as the medical image diagnostic apparatus of a simple X-ray apparatus, an X-ray fluoroscopy apparatus, an X-ray computed tomography (CT) apparatus, a magnetic resonance imaging (MRI) apparatus, a nuclear medicine diagnostic apparatus or the like. Further, the image processing apparatus according to any of the embodiments may be installed separately from the medical image diagnostic apparatus that processes the medical image data acquired by the medical image diagnostic apparatus. Hereinafter, in the second embodiment, a case where the image processing apparatus is installed separately from the medical image diagnostic apparatus will be described.

### First Embodiment

An image processing apparatus according to the first embodiment is provided as a medical image diagnostic apparatus of an ultrasonic diagnostic apparatus.

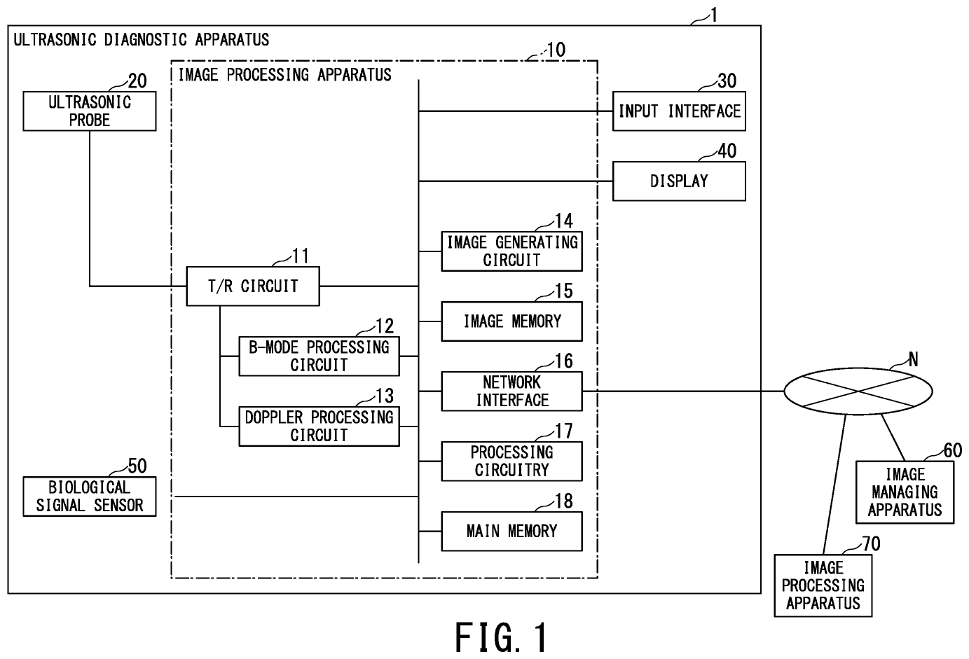

FIG. 1 is a schematic view showing an example of a configuration of the ultrasonic diagnostic apparatus provided with the image processing apparatus according to the first embodiment.

FIG. 1 shows an ultrasonic diagnostic apparatus 1 including an image processing apparatus 10 according to the first embodiment. As shown in FIG. 1, the ultrasonic diagnostic apparatus 1 includes an ultrasonic probe 20, an input interface 30, a display 40, and a biological signal sensor 50 in addition to the image processing apparatus 10.

Note that an apparatus in which at least one of the ultrasonic probe 20, the input interface 30, the display 40 and the biological signal sensor 50 being added to the image processing apparatus 10 may be referred to as “image processing apparatus”. In the following description, a case will be described where the ultrasonic probe 20, the input interface 30, the display 40 and the biological signal sensor 50 are all provided outside the image processing apparatus 10.

The image processing apparatus 10 includes a transmitting/receiving (T/R) circuit 11, a B-mode processing circuit 12, a Doppler processing circuit 13, an image generating circuit 14, an image memory 15, a network interface 16, processing circuitry 17, and a main memory 18. The circuits 11 to 14 are configured by application-specific integrated circuits (ASICs) and the like. However, the present invention is not limited to this case, and all or part of the functions of the circuits 11 to 14 may be realized by the processing circuitry 17 executing a program.

The T/R circuit 11 has a transmitting circuit and a receiving circuit (both not shown). Under the control of the processing circuitry 17, the T/R circuit 11 controls transmission directivity and reception directivity in transmitting and receiving ultrasonic waves. The case where the T/R circuit 11 is provided in the image processing apparatus 10 will be described, but the T/R circuit 11 may be provided in the ultrasonic probe 20, or may be provided in both of the image processing apparatus 10 and the ultrasonic probe 20. The T/R circuit 11 is an example of a transmitting/receiving unit.

The transmitting circuit, which has a pulse generating circuit, a transmission delay circuit, a pulsar circuit, and the like, sends a drive signal to ultrasonic transducers of the ultrasonic probe 20. The pulse generating circuit repeatedly generates rate pulses for forming transmission ultrasonic waves at a predetermined rate frequency. The transmission delay circuit converges the ultrasonic waves generated from the ultrasonic transducer of the ultrasonic probe 20 into a beam shape, and gives a delay time of each piezoelectric transducer necessary for determining the transmission directivity to each rate pulse generated by the pulse generating circuit. The pulsar circuit applies drive pulses to each ultrasonic transducer at a timing based on the rate pulses. The transmission delay circuit arbitrarily adjusts the transmission direction of the ultrasonic beam transmitted from a piezoelectric transducer surface by changing the delay time given to each rate pulse.

The receiving circuit, which has an amplifier circuit, an analog to digital (A/D) converter, an adder, and the like, receives the echo signal received by the ultrasonic transducers, and generate echo data by performing various processes on the echo signal. The amplifier circuit amplifies the echo signal for each channel, and performs gain correction processing. The A/D converter A/D-converts the gain-corrected echo signal, and gives a delay time necessary for determining the reception directivity to the digital data. The adder adds the echo signal processed by the A/D converter to generate echo data. By the addition processing of the adder, the reflection component from the direction corresponding to the reception directivity of the echo signal is emphasized.

Under the control of the processing circuitry 17, the B-mode processing circuit 12 receives the echo data from the receiving circuit, performs logarithmic amplification, envelope detection processing and the like, thereby generating data (two-dimensional (2D) or three-dimensional (3D) data) which signal intensity is represented by brightness of luminance. This data is generally called “B-mode data”. The B-mode processing circuit 12 is an example of a B-mode processing unit.

The B-mode processing circuit 12 may change the frequency band to be visualized by changing the detection frequency using filtering processing. By using the filtering processing function of the B-mode processing circuit 12, harmonic imaging such as the contrast harmonic imaging (CHI) or the tissue harmonic imaging (THI) is performed. That is, the B-mode processing circuit 12 may separate the reflected wave data of a subject where the contrast agent is injected into harmonic data (or sub-frequency data) and fundamental wave data. The harmonic data (or sub-frequency data) refers to the reflected wave data having a harmonic component whose reflection source is the contrast agent (microbubbles or bubbles) in the subject. The fundamental wave data refers to the reflected wave data having a fundamental wave component whose reflection source is tissue in the subject. The B-mode processing circuit 12 is able to generate B-mode data for generating contrast image data based on the reflected wave data (received signal) having the harmonic component, and to generate B-mode data for generating fundamental wave image data based on the reflected wave data (received signal) having the fundamental wave component.

In the THI using the filtering processing function of the B-mode processing circuit 12, it is possible to separate harmonic data or sub-frequency data, which is reflected wave data (received signal) having a harmonic component, from reflected wave data of the subject. Then, the B-mode processing circuit 12 generates B-mode data for generating tissue image data in which the noise component is removed from the reflected wave data (received signal) having the harmonic component.

When the CHI or THI harmonic imaging is performed, the B-mode processing circuit 12 may extract the harmonic component by a method different from the method using the above-described filtering. In the harmonic imaging, an amplitude modulation (AM) method, a phase modulation (PM) method, or an AM-PM method combining the AM method and the PM method is performed. In the AM method, the PM method, and the AM-PM method, ultrasonic transmission with different amplitudes and phases is performed multiple times on the same scanning line. Thereby, the T/R circuit 11 generates and outputs multiple reflected wave data (received signal) in each scanning line. The B-mode processing circuit 12 extracts harmonic components out of the multiple reflected wave data (received signal) of each scanning line by performing addition/subtraction processing according to the modulation method. The B-mode processing circuit performs envelope detection processing etc. on the reflected wave data (received signal) having the harmonic component to generate B-mode data.

For example, when the PM method is performed, the T/R circuit 11 transmits the ultrasonic waves of the same amplitude and reversed-phase polarities, such as (−1, 1), twice by each scanning line in a scan sequence set by the processing circuitry 17. The T/R circuit 11 generates a reception signal based on transmission of “−1” and a reception signal based on transmission of “1”. The B-mode processing circuit 12 adds these two reception signals. As a result, a signal in which the fundamental wave component is removed while the second harmonic component mainly remains is generated. Then, the B-mode processing circuit 12 performs envelope detection processing and the like on this signal to generate B-mode data using THI or CHI.

Alternatively, for example, in the THI, an imaging method using the second harmonic component and a difference tone component included in the received signal has been put into practice. In the imaging method using the difference tone component, the transmission ultrasonic waves having, for example, a composite waveform combining a first fundamental waves with a center frequency “f1” and a second fundamental waves with a center frequency “f2” larger than “f1” are transmitted from the ultrasonic probe 20. Such a composite waveform combines a wave form of the first fundamental waves and a waveform of the second fundamental waves whose phases are adjusted with each other, such that the difference tone component having the same polarity as the second harmonic component is generated. The T/R circuit 11 transmits the transmission ultrasonic waves having the composite waveform, for example, twice while inverting the phase. In such a case, for example, the B-mode processing circuit 12 performs an envelope detection processing etc. after extracting a harmonic component in which the fundamental wave component is removed by adding two received signals while the difference tone component and the second harmonic component are mainly left.

Under the control of the processing circuitry 17, the Doppler processing circuit 13 frequency-analyzes the phase information based on the echo data from the receiving circuit, thereby generating data (2D or 3D data) by extracting multiple moving data of a moving subject such as average speed, dispersion, power, and the like. This data is an example of the raw data, and is generally called “Doppler data”. In the specification, the moving subject refers to, for example, blood flow, tissue such as heart wall, or contrast agent. The Doppler processing circuit 13 is an example of a Doppler processing unit.

Under the control of the processing circuitry 17, the image generating circuit 14 generates an ultrasonic image shown in a predetermined luminance range as image data based on the echo signal received by the ultrasonic probe 20. For example, the image generating circuit 14 generates a B-mode image as an ultrasonic image in which the intensity of the reflected wave is represented by luminance based on the two-dimensional B-mode data generated by the B-mode processing circuit 12. Further, the image generating circuit 14 generates, as an ultrasonic image, an average velocity image representing movement information, a distributed image, a power image, or a color Doppler image as a combination image thereof based on the two-dimensional Doppler data generated by the Doppler processing circuit 13. The image generating circuit 14 is an example of an image generating unit.

In the present embodiment, the image generating circuit 14 generally converts (scan-converts) a scanning line signal sequence of ultrasonic scanning into a scanning line signal sequence of a video format used by a television or the like, and generates ultrasonic image data for display. Specifically, the image generating circuit 14 generates ultrasonic image data for display by performing coordinate conversion according to the ultrasonic scanning mode of the ultrasonic probe 20. The image generating circuit 14 performs various image processes other than the scan conversion. For example, the image generating circuit 14 performs image processing (smoothing processing) for regenerating an average luminance image using multiple image frames after scan conversion, image processing (for enhancing edges) using a differential filter in the image and the like. Further, the image generating circuit 14 combines character information of various parameters, scales, body marks, and the like with the ultrasonic image data.

That is, the B-mode data and the Doppler data are the ultrasonic image data before the scan conversion processing. The data generated by the image generating circuit 14 is ultrasonic image data for display after the scan conversion processing. The B-mode data and the Doppler data are also called raw data. The image generating circuit 14 generates two-dimensional ultrasonic image data for display based on the two-dimensional ultrasonic image data before the scan conversion processing.

Further, the image generating circuit 14 performs coordinate conversion on the three-dimensional B-mode data generated by the B-mode processing circuit 12, thereby generates three-dimensional B-mode image data. The image generating circuit 14 performs coordinate conversion on the three-dimensional Doppler data generated by the Doppler processing circuit 13, thereby generates three-dimensional Doppler image data. The image generating circuit 14 generates “three-dimensional B-mode image data or three-dimensional Doppler image data” as “three-dimensional ultrasonic image data (volume data)”.

Further, the image generating circuit 14 performs a rendering processing on the volume data to generate various two-dimensional image data for displaying the volume data on the display 40. The image generating circuit 14 performs, for example, an MPR processing as the rendering processing that generates a multi planer reconstruction (MPR) image data based on the volume data. Further, the image generating circuit 14 performs, for example, a volume rendering (VR) processing as the rendering processing that generates two-dimensional image data reflecting three-dimensional data.

The image memory 15 is, for example, a magnetic or optical recording medium, a recording medium that can be read by a processor such as a semiconductor memory, or the like. The image memory 15 stores ultrasonic image data of multiple heartbeats associated with the heartbeat data under the control of the processing circuitry 17 and generated by the image generating 14. The multiple ultrasonic image data stored in the image memory 15 are associated with the heartbeat data of the subject in the unit of one heartbeat (one cardiac cycle). Specifically, for example, each ultrasonic image data stored in the image memory 15 is associated with heartbeat data corresponding to one heartbeat.

The image memory 15 may store multiple ultrasonic image data of one heartbeat as one image data, or multiple ultrasonic image data of multiple heartbeats may be collectively stored in one image data. Further, the image memory 15 may store the ultrasonic image data generated by the image generating circuit 14 not only as two-dimensional data but also as volume data under the control of the processing circuitry 17. The image memory 15 is an example of a storage unit.

The network interface 16 implements various information communication protocols according to the network form. The network interface 16 connects the ultrasonic diagnostic apparatus 1 and other devices such as the external image managing apparatus 60 and the image processing apparatus 70 according to these various protocols. An electrical connection or the like via an electronic network is applied to this connection. In the present embodiment, the electronic network means an entire information communication network using telecommunications technology. The electronic network includes a wired/wireless hospital backbone local area network (LAN) and the Internet network, as well as a telephone communication line network, an optical fiber communication network, a cable communication network, a satellite communication network, or the like.

Further, the network interface 16 may implement various protocols for non-contact wireless communication. In this case, the image processing apparatus 10 can directly transmit/receive data to/from the ultrasonic probe 20, for example, without going through the network. The network interface 16 is one example of a network connector.

The processing circuitry 17 may be a processor such as a dedicated or general-purpose CPU (central processing unit), an MPU (microprocessor unit), a GPU (Graphics Processing Unit), or the like. The processing circuitry 17 may be an ASIC, a programmable logic device, or the like. The programmable logic device is, for example, a simple programmable logic device (SPLD), a complex programmable logic device (CPLD), and a field programmable gate array (FPGA).

Further, the processing circuitry 17 may be constituted by a single circuit or a combination of independent circuit elements. In the latter case, the main memory 18 may be provided individually for each circuit element, or a single main memory 18 may store programs corresponding to the functions of the circuit elements. The processing circuitry 17 is an example of a processor.

The main memory 18 is constituted by a semiconductor memory element such as a random-access memory (RAM), a flash memory, a hard disk, an optical disk, or the like. The main memory 18 may be constituted by a portable medium such as a universal serial bus (USB) memory and a digital video disk (DVD). The main memory 18 stores various processing programs (including an operating system (OS) and the like besides the application program) used in the processing circuitry 17 and data necessary for executing the programs. In addition, the OS may include a graphical user interface (GUI) which enables the display 40 to frequently display information using graphics to the operator, and allows the operator to perform basic operations by the input interface 30. The main memory 18 is an example of a storage unit.

The ultrasonic probe 20 includes microscopic transducers (piezoelectric elements) on the front surface portion that transmits and receives ultrasonic waves to and from a region including a scan target, such as a region including a lumen. Each transducer is an electroacoustic transducer, and has a function of converting electric pulses into ultrasonic pulses at the time of transmission and converting reflected waves into electric signals (reception signals) at the time of reception. The ultrasonic probe 20 is configured to be small and lightweight, and is connected to the ultrasonic diagnostic apparatus 10 via a cable (or wireless communication).

The ultrasonic probe 20 is classified into a linear type, a convex type, a sector type, etc. depending on differences in scanning system. Further, the ultrasonic probe 20 is classified into a 1D array probe in which transducers are arrayed in a one-dimensional (1D) manner in the azimuth direction, and a 2D array probe in which transducers are arrayed in a two-dimensional (2D) manner in the azimuth direction and the elevation direction, depending on the differences in array arrangement dimension. The 1D array probe also includes a probe in which a small number of transducers are arranged in the elevation direction.

In the present embodiments, when a three-dimensional (3D) scan, that is, a volume scan is executed, the 2D array probe having a scan type such as the linear type, the convex type, the sector type, or the like is used as the ultrasonic probe 20. Alternatively, when the volume scan is executed, the 1D probe having a scan type such as the linear type, the convex type, the sector type etc., and having a mechanism that mechanically oscillates in the elevation direction is used as the ultrasonic probe 20. The latter probe is also called a mechanical 4D probe.

The input interface 30 includes an input device operable by an operator, and a circuit enables signal input from the input device. The input device may be a trackball, a switch, a mouse, a keyboard, a touch pad where an input operation is performed by touching an operation surface, a touch screen in which a display screen and a touch pad are integrated, a non-contact input circuit using an optical sensor, an audio input circuit, and the like. When the input device is operated by the operator, the input interface 30 generates an input signal corresponding to the operation and outputs it to the processing circuitry 17. The input interface 30 is an example of an input unit.

The display 40 is constituted by a general display output device such as a liquid crystal display or an organic light emitting diode (OLED) display. The display 40 displays various kinds of information under the control of the processing circuitry 17. The display 40 is an example of a display unit.

The biological signal sensor 50 detects a biological signal from the subject to be ultrasonically scanned. The biological signal sensor 50 detects, for example, an electrocardiogram (ECG) signal of the subject as an electrical signal. The biological signal sensor 50 performs various processes including digitizing the detected ECG signal, and then transmitting the detected ECG signal to the image processing apparatus 10 as heartbeat data. In addition to/instead of ECG, the biological signal sensor 50 may detect other signal having periodicity such as brain waves, pulse, and respiration that are emitted from the subject.

FIG. 1 shows the image managing apparatus 60 and the image processing apparatus 70 which are external devices of the image processing apparatus 10. The image managing apparatus 60 is, for example, a digital imaging and communications in medicine (DICOM) server, and is connected to a device such as the ultrasonic diagnostic apparatus 1 such that data can be transmitted and received via the network N. The image managing apparatus 60 manages a medical image, such as an ultrasonic image generated by the ultrasonic diagnostic apparatus 1, as the DICOM file.

The image processing apparatus 70 is connected to devices such as the ultrasonic diagnostic apparatus 1 and the medical image managing apparatus 60 such that data is transmitted and received via the network N. The image processing apparatus 70 may be a workstation that performs various image processing on the ultrasonic image generated by the ultrasonic diagnostic apparatus 1, a portable information processing terminal such as a tablet terminal, etc. It should be noted that the image processing apparatus 70 is an offline apparatus and may be an apparatus capable of reading an ultrasonic image generated by the ultrasonic diagnostic apparatus 1 via a portable storage medium.

Subsequently, functions of the image processing apparatus 10 will be described.

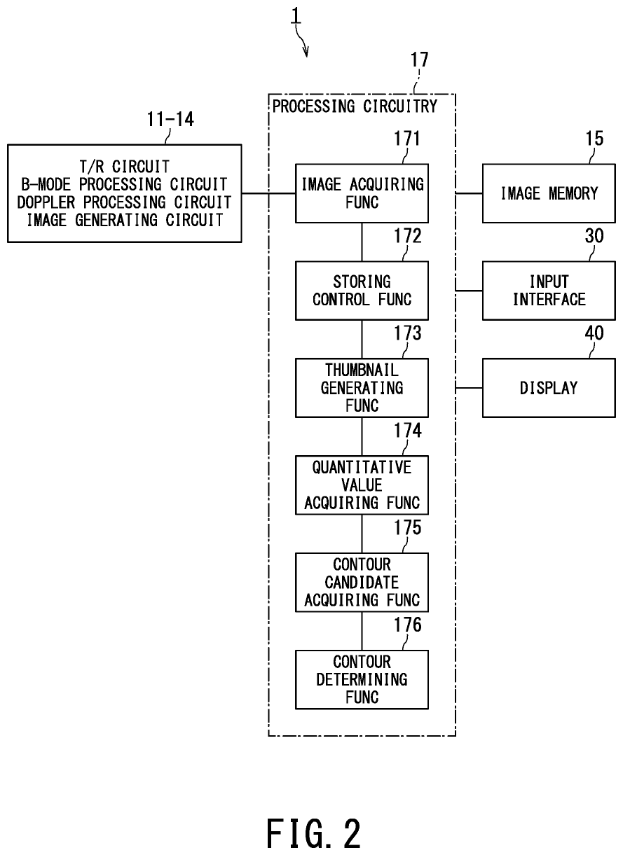

FIG. 2 is a block diagram showing an example of functions of the image processing apparatus 10.

The processing circuitry 17 reads out and executes a computer program (e.g., an image processing program) stored in the main memory 18 or a memory in the processing circuitry 17, thereby realizing an image acquiring function 171, a storing control function 172, a thumbnail generating function 173, a quantitative value acquiring function 174, a contour candidate acquiring function 175, and a contour determining function 176. Hereinafter, the case where the functions 171 to 176 are realized by a computer program will be described as an example, but the present invention is not limited to this case. All or part of the functions 171 to 176 may be provided in the image processing apparatus 10 as a function of a circuit such as an ASIC.

Here, in the echocardiography by an ultrasonic diagnostic apparatus, after performing freeze operation on the displayed ultrasonic image, the operator manually selects the most suitable heartbeat out of the latest multiple heartbeats. Then, various measurements and analyzes are performed on the ultrasonic image corresponding to the selected heartbeat to acquire a quantitative value indicating the cardiac function. Examples of the first method for acquiring quantitative values indicating the cardiac function include two-dimensional or three-dimensional WMT (Wall Motion Tracking) for analyzing the wall motion of the myocardium, and “Auto EF” (Automated Ejection Fraction) that automatically calculates the left ventricular ejection fraction and the like. The “Auto EF” is a method of collating an actual heart morphology with features registered in a pre-constructed database (heart appearance, left ventricular endocardium, etc.) to perform pattern recognition, searching for a heart having a similar pattern, detecting (tracing) the left ventricular endocardium, and then calculating the end-diastolic volume (EDV) of left-ventricular, the end-systolic volume (ESV) of left-ventricular, the ejection fraction (EF) of left-ventricular or the like as a quantitative value of each heartbeat. According to the first method of measuring and analyzing an ultrasonic image, the contour is traced without information preset by an operator such as a doctor. Therefore, not only the trace result but also the quantitative value such as EF may be different from what the operator desires.

Further, as the second method for acquiring the quantitative value indicating the cardiac function, there is a technique of generating a database by associating the ultrasonic image data with the quantitative value in advance, and referring to a database to acquire a quantitative value corresponding to desired ultrasonic image data. For example, machine learning is used in the process of acquiring a quantitative value indicating the cardiac function. Further, deep learning using a multi-layer neural network such as a convolutional neural network (CNN), a convolutional deep belief network (CDBN), or the like is applied as machine learning. According to the second method of measuring and analyzing an ultrasonic image, the acquired quantitative value may be different from the quantitative value acquired from the contour of the same ultrasonic image. Further, according to the second method, the ultrasonic image from which the ejection fraction (EF) or the like is acquired does not remain as a basis but disappear. Therefore, the operator cannot correct the contour trace result that should be performed on the ultrasonic image at later stage (e.g., before and after the surgical operation).

Therefore, the processing circuitry 17 realizes the functions 171 to 176 described later.

The image acquiring function 171 includes a function of controlling the T/R circuit 11, the B-mode processing circuit 12, the Doppler processing circuit 13, the image generating circuit 14, etc., and acquiring ultrasonic image data by ultrasonic scanning using the ultrasonic probe 20.

Specifically, the image acquiring function 171 acquires M-mode image data, B-mode image data, Doppler image data, and the like as the ultrasonic image data. Further, the image acquiring function 171 includes a function of displaying each live ultrasonic image on the display 40 based on the ultrasonic image data generated by the image generating circuit 14. The image acquiring function 171 is an example of an image acquiring unit.

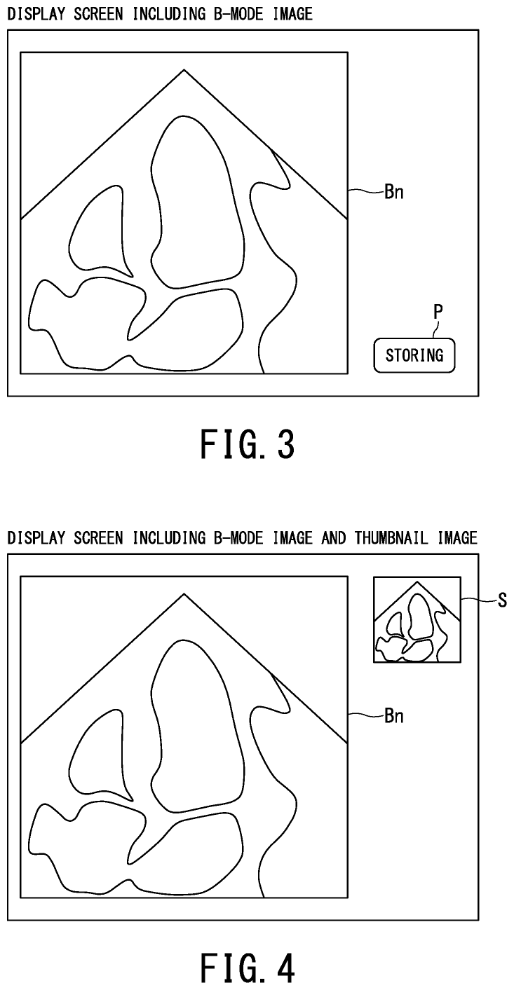

FIG. 3 is a diagram showing a display example of the B-mode image.

FIG. 3 shows a display screen including the B-mode image as the ultrasonic image. This display screen includes the B-mode image Bn of the n-th frame (moving image data) to be displayed as live image. The “n” is an integer of 1 or more. The B-mode image Bn+1 of the (n+1)-th frame is superimposed on the B-mode image Bn of the n-th frame, and the display of the B-mode image is updated, so that the B-mode image is displayed as live image. Although not shown in FIG. 3, the display screen may show heartbeat data (e.g., an electrocardiogram waveform).

Returning to the description of FIG. 2, the storing control function 172 includes a function of acquiring heartbeat data output from the biological signal sensor 50, and sequentially storing (primarily storing) ultrasonic image data of multiple frames generated by the image generating circuit 14 associated with the heartbeat data in the image memory 15. The storing control function 172 may sequentially store the ultrasonic image data in the image memory 15 while sequentially storing the heartbeat data associated with the ultrasonic image data in another memory (not shown). Further, the storing control function 172 may acquire heartbeat data of multiple heartbeats of the subject while the ultrasonic image data are acquired.

Further, the storing control function 172 includes a function of storing (secondarily storing) the ultrasonic image data of the multiple frames corresponding to ultrasonic image data of a specific frame in the image memory 15 as moving image data. The specific frame relates to a storing instruction received by the input interface 30. The storing control function 172 is an example of the storing control unit.

The thumbnail generating function 173 includes a function of generating thumbnail image data indicating a thumbnail of the ultrasonic image data of the specific frame according to the storing instruction received by the input interface 30. Further, the thumbnail generating function 173 includes a function of displaying thumbnail image data as a thumbnail image on the display 40. The thumbnail generating function 173 is an example of a generating unit.

FIG. 4 is a diagram showing a display example of the thumbnail image.

FIG. 4 shows a display screen including the thumbnail image. This display screen includes a B-mode image Bn of the n-th frame to be displayed as live image, and a thumbnail image S indicating a thumbnail of the B-mode image data stored as instructed.

In the display screen shown in FIG. 3, a marker is aligned with a “STORING” button P by the input interface 30 and determined (clicked). And then, the thumbnail generating function 173 shifts the display screen shown in FIG. 3 to the display screen shown in FIG. 4.

Returning to the description of FIG. 2, the quantitative value acquiring function 174 includes a function of acquiring a quantitative value (for example, XX %) indicating a function of a tissue such as the heart, acquiring multiple contour candidates based on the quantitative value, superimposing the multiple contour candidates on the ultrasonic image data, and displaying them on the display 40. The quantitative value acquiring function 174 may acquire the quantitative value by a technique such as “Auto EF” described above, or may acquire the quantitative value by deep learning using the database or the multi-layer neural network described above. Further, the quantitative value acquiring function 174 can also acquire a numerical value manually input from the input interface 30 as the quantitative value. In that case, the operator can input the quantitative value while referring to the moving image data according to the storing instruction of the input interface 30. The quantitative value acquiring function 174 is an example of the quantitative value acquiring unit.

In addition, the quantitative value acquiring function 174 can also change the acquired quantitative value by changing the selected heartbeat or changing the selected end-diastolic or end-systolic frame. As a result, the quantitative value can be changed without modifying the contour data described later.

The contour candidate acquiring function 175 includes a function of acquiring, for example, information of multiple contour candidates of the left ventricular endocardium of a heart based on the quantitative value acquired by the quantitative value acquiring function 174. The contour information includes the position and shape of the contour of the left ventricular endocardium. Further, the contour candidate acquiring function 175 includes a function of displaying the processing result on the display 40 when the processing of acquiring the contour candidates is completed. The contour candidate acquiring function 175 is an example of the contour candidate acquiring unit.

The contour determining function 176 includes a function of determining contour data selected by the input interface 30 out of the multiple contour candidates displayed by the contour candidate acquiring function 175. The determined contour data corresponds to the moving image data according to the storing instruction of the input interface 30. The contour determining function 176 is an example of the contour determining unit.

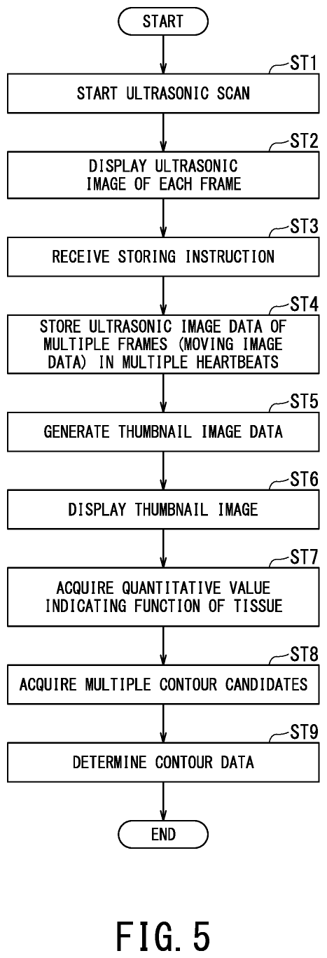

The details of the functions 171 to 176 will be described with reference to FIGS. 5 to 9.

Subsequently, an operation of the image processing apparatus 10 will be described.

FIG. 5 is a flowchart showing an example of the operation of the image processing apparatus 10 a. In FIG. 5, the reference numeral “ST” with a number indicates each step of the flowchart.

The image acquiring function 171 of the image processing apparatus 10 receives examination order information from an examination requesting apparatus (not shown) such as HIS (Hospital Information Systems), and then receives instructions to start an ultrasonic scan of the echocardiography via the input interface 30. The image acquiring function 171 controls the T/R circuit 11, the B-mode processing circuit 12, the Doppler processing circuit 13, the image generating circuit 14, and the like, thereby starting the ultrasonic scan using the ultrasonic probe 20 (step ST1). The image acquiring function 171 displays the B-mode image data of each frame including the heart acquired in step ST1 on the display 40 as a live B-mode image (step ST2). The example of displaying the B-mode image is shown in FIG. 3.

The storing control function 172 receives an instruction from the input interface 30 to store the B-mode image data of the frame displayed on the display 40 by step ST2 (step ST3). Generally, update B-mode image displayed as live image is frozen before receiving the storing instruction from the input interface 30.

The storing control function 172, based on the storing instruction received in ST3, acquires B-mode image data of multiple frames in multiple heartbeats immediately before the storing instruction, and stores the B-mode image data as moving image data in the image memory 15 (step ST4). For example, in step ST4, the storing control function 172 acquires B-mode image data of multiple frames in four heartbeats immediately before the storing instruction among the B-mode image data of the multiple frames that are primarily stored in the image memory 15 (or the main memory 18), and secondarily stores them in the image memory 15. The multi-frame B-mode image data primarily stored in the image memory 15 are associated with the heartbeat data.

The thumbnail generating function 173 generates thumbnail image data indicating a thumbnail of the B-mode image data that have been stored according to the storing instruction received in step ST3 (step ST5). The thumbnail generating function 173 displays the live B-mode image data, and displays the thumbnail image data generated in step ST5 on the display 40 as a thumbnail image (step ST6). The display example of the thumbnail image is shown in FIG. 4.

The quantitative value acquiring function 174 acquires a quantitative value (e.g., XX %) indicating the function of a tissue, such as the heart (step ST7). For example, the quantitative value acquiring function 174 acquires a quantitative value by a technique such as “Auto EF” described above. Alternatively, the quantitative value acquiring function 174 acquires the quantitative value by deep learning using the database or the multi-layer neural network described above. Further, the quantitative value acquiring function 174 can also acquire a numerical value manually input from the input interface 30 as a quantitative value. In that case, the operator can input the quantitative value while referring to the moving image data related to the storing instruction received by the input interface 30. In addition, the quantitative value acquiring function 174 can also change the acquired quantitative value by changing the selected heartbeat or changing the selected end-diastolic or end-systolic frame.

The contour candidate acquiring function 175 acquires contour candidates based on the quantitative values acquired in step ST7 (step ST8).

In step ST8, the contour candidate acquiring function 175 may use the database, where each quantitative value corresponds to the contour for example, for the processing of acquiring the contour candidates of the left ventricular endocardium based on the quantitative value acquired by the quantitative value acquiring function 174. Further, the contour candidate acquiring function 175 may use machine learning for the processing of acquiring the contour candidates based on the quantitative value acquired by the quantitative value acquiring function 174. Further, as machine learning, deep learning using a multi-layer neural network may be used.

Hereinafter, an example will be shown in which the contour candidate acquiring function 175 includes a neural network Na and uses deep learning to acquire contour candidates of the left ventricular endocardium based on the quantitative value indicating the function of the heart.

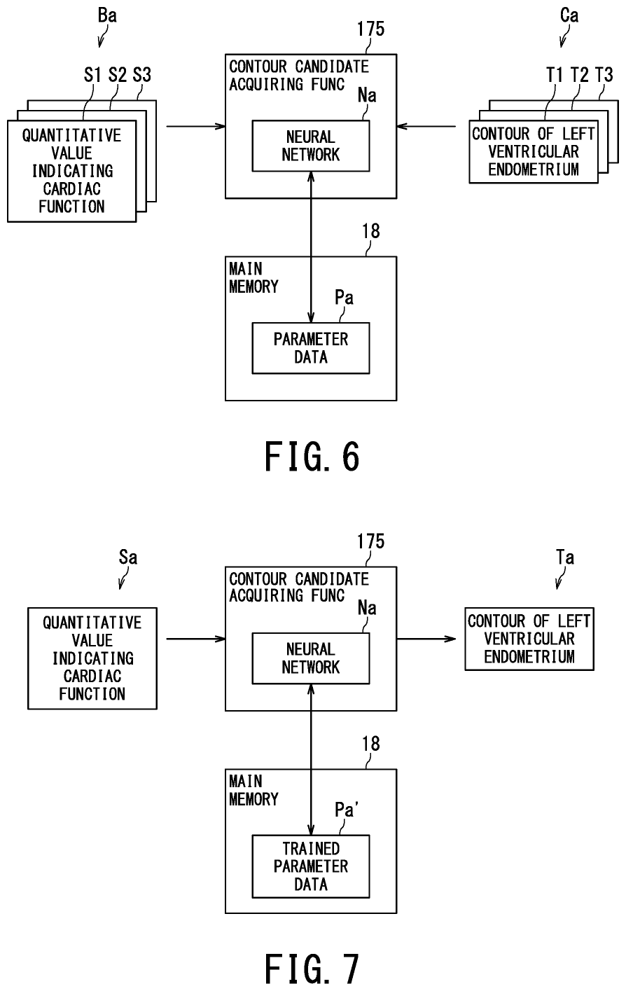

FIG. 6 is an explanatory diagram showing an example of a data flow during learning.

The contour candidate acquiring function 175 sequentially updates the parameter data Pa by a large number of input training data and learning. The input training data includes quantitative values S1, S2, S3, . . . indicating the cardiac function (e.g., quantitative values indicating EF) and contours T1, T2, T3, . . . of the endocardium (e.g., the left ventricular endocardium). The quantitative values S1, S2, S3, . . . constitute the input training data group Ba. The contours T1, T2, T3, . . . constitute the output training data group Ca.

The contour candidate acquiring function 175 updates the parameter data Pa each time the training data is input so that the result of processing the quantitative values S1, S2, S3, by the neural network Na approaches the contours T1, T2, T3, which is so-called learning. Generally, when the rate of change of the parameter data Pa converges within the threshold value, it is determined that the learning is completed. Hereinafter, the parameter data Pa after learning is particularly referred to as trained parameter data Pa′.

Note that the type of input training data and the type of input data during operation shown in FIG. 7 should be the same. For example, when the input data at the time of operation is a quantitative value, the input training data group Ba at the time of learning is also used as the quantitative value.

FIG. 7 is an explanatory diagram showing an example of a data flow during operation.

At the time of operation, the contour candidate acquiring function 175 inputs a quantitative value Sa indicating the cardiac function acquired in step ST7, and outputs contour Ta of the left ventricular endocardium using the learned parameter data Pa′.

The neural network Na and the trained parameter data Pa′ constitute the trained model 19a. The neural network Na is stored in the main memory 18 in the form of a program. The trained parameter data Pa′ may be stored in the main memory 18 or may be stored in a storage medium connected to the ultrasonic diagnostic apparatus 1 via the network N. In this case, the contour candidate acquiring function 175, which is realized by the processor of the processing circuitry 17, readouts the trained model 19a from the main memory 18 and executes it so as to generate contour data. The trained model 19a may be constructed by an integrated circuit such as an ASIC (Application Specific Integrated Circuit) or an FPGA (Field Programmable Gate Array).

In addition to the quantitative value, supplementary information including at least one of the height or weight of the object to be imaged, image data of other already-imaged modality, and representative model data of the gadget may be used as the input data so as to improve the judgment accuracy of the contour candidate acquiring function 175.

In this case, at the time of learning, supplementary information of each subject is also input to the neural network Na as input training data like the quantitative values S1, S2, S3, . . . . At the time of operation, the contour candidate acquiring function 175 inputs supplementary information of the subject to be imaged together with the acquired quantitative value Ba into the trained model 19a readout from the main memory 18, and outputs contour Ta. By using the quantitative value and the supplementary information of the object to be imaged as the input data, it is possible to generate the trained parameter data Pa′ that has been trained according to the type of the subject.

Therefore, the acquisition accuracy can be improved as compared with the case using only the quantitative value as the input data.

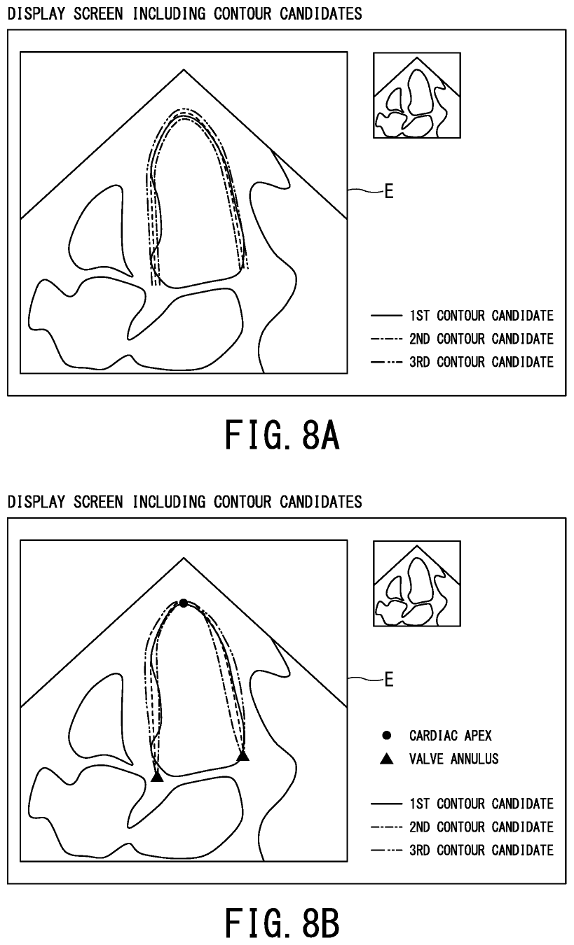

The contour candidate acquiring function 175 acquires one or more contours with high accuracy by deep learning using the above-mentioned database or a multi-layer neural network, and displays the acquired contour as contour candidates on the display 40. For example, the contour candidate acquiring function 175 acquires the most accurate one contour based on the ultrasonic image as the contour candidate. Alternatively, the contour candidate acquiring function 175 acquires multiple contours with high accuracy as the contour candidates (shown in FIG. 8A). Alternatively, the contour candidate acquiring function 175 acquires the most accurate one contour, and further acquires multiple contours as the contour candidates based on the most accurate one contour (shown in FIG. 8B).

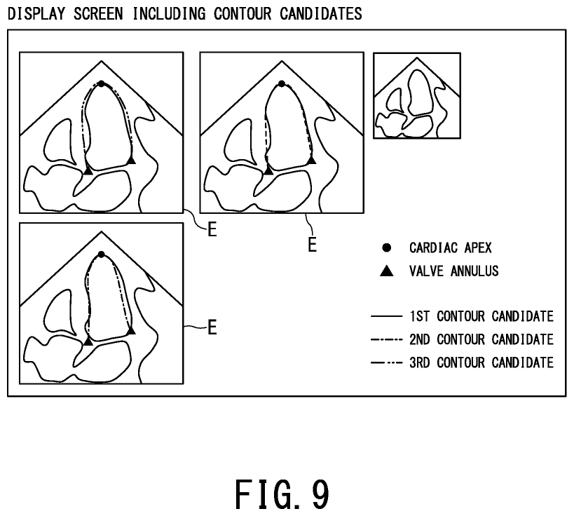

Each of FIGS. 8A, 8B and 9 are diagrams showing an example of a display screen including contour candidates. FIG. 8A shows the first display screen including contour candidates. FIG. 8B shows the second display screen including contour candidates. FIG. 9 shows the third display screen including contour candidates. The case where the number of the contour candidates is three will be described, but it is not limited to that case. The number of the contour candidates may be two or more.

FIG. 8A shows, when the most accurate three contours are acquired as contour candidates (1st to 3rd candidates) based on the ultrasonic image, a display screen in which three contour candidates are superimposed on one end-diastole image data (or moving image data or end-systole image data) E. As shown in FIG. 8A, three contour candidates can be represented by different line types (solid line, broken line, thick line, etc.). Alternatively, the three contour candidates can be expressed in different colors (hue, saturation, lightness). In addition, the frame of the displayed end-diastole image data or the end-systole image data can be changed arbitrarily.

FIG. 8B shows, when one contour with the highest accuracy based on the ultrasonic image and the other two contours whose feature points match a feature point of the one contour are acquired as contour candidates (1st to 3rd candidates), a display screen in which three contour candidates are superimposed on one expansion end image data (or moving image data or end contraction image data) E. For example, at least one of image data corresponding to the frame at the end of expansion and image data corresponding to the frame at the end of contraction among the moving image data stored by the storing instruction is acquired. The annulus portion and the apex portion which are the feature points are fixed to the image data, and the contour is enlarged or reduced as a whole. Then, the other two contour candidates are acquired and displayed. Alternatively, by fixing the annulus portion and the apex of the heart to the image data and enlarging or reducing a part of the contour, other contour candidates are acquired and displayed. Alternatively, by moving the feature point such as the annulus portion and the apex of the heart with respect to the image data, the other two contour candidates are acquired and displayed. In addition, the frame of the displayed image data at the end of expansion and the end of contraction can be changed arbitrarily.

FIG. 9 shows, when one contour with the highest accuracy based on the ultrasonic image and the other two contours whose feature points match a feature point of the one contour are acquired as contour candidates (1st to 3rd candidates), a display screen in which an image in which one candidate for contour is superimposed on one image data at the end of expansion (or moving image data or image data at the end of contraction) E is arranged in parallel. In addition, the frame of the displayed image data at the end of expansion and the end of contraction can be changed arbitrarily.

The contour candidate acquiring function 175 reacquires and displays multiple contour candidates of the left ventricular endocardium according to the adjustment of the quantitative value. Further, the contour candidate acquiring function 175 can preset the number of the contour candidates. Also, the contour candidate acquiring function 175 preset a feature point such as the annulus or the apex of the heart, and acquires contour candidates that pass the feature point. In such manner, the contour candidate acquiring function 175 narrows down the options and shortens the time of selecting a desired contour for the operator. Further, due to the limitation of the quantitative value and the feature point, it may be difficult to acquire the preset number of contour candidates. In that case, the contour candidate acquiring function 175 may display a “warning” sign on the display screen, or recommend changing the quantitative value, changing (reducing) the preset number of contour candidates, or recommend changing (adjusting) the feature points via the display screen. In that case, the contour candidate acquiring function 175 may accept changes in the quantitative value, changes (reduction) in the preset number of contour candidates, or changes (adjustments) of feature points via the input interface 30.

The contour determining function 176 determines a selected contour, which is chosen from the contour candidates displayed by the contour candidate acquiring function 175 via the input interface 30, as contour data to be associated with the moving image data stored according to the storing instruction in step ST3 (step ST9). The contour determining function 176 can also adjust the selected contour chosen from the contour candidates displayed by the contour candidate acquiring function 175 based on the input information from the input interface 73. For example, the contour candidate acquiring function 175 can adjust the contour by adjusting the annulus or the apex of the heart on the image shown in FIG. 8B. Alternatively, the contour candidate acquiring function 175 can make adjustments by enlarging or contracting the overall contour shown in FIG. 8B.

In the above, an example of adopting the EF of the heart as the quantitative value indicating the cardiac function of each heartbeat has been described. However, the present invention can also be applied to quantitative values other than EF. For example, when the tissue is the heart, the end-diastolic volume (EDV) of left-ventricular, the end-systolic volume (ESV) of left-ventricular, the ejection fraction (EF) of left-ventricular, the global longitudinal strain (GLS), the fractional area change (FAC) of right-ventricular, or the like may be adopted as a quantitative value indicating the cardiac function of each heartbeat. When the tissue is a blood vessel, the quantitative value indicating the function of the blood vessel is, for example, the stenosis rate.

According to the image processing apparatus 10 in the first embodiment, when the quantitative value such as EF is acquired by using the “Auto EF” function, contour candidates corresponding to the quantitative value can be presented to the operator, and the operator can select desired contour data out of the candidates. As a result, it is possible to acquire contour data that is desirable and also corresponding to the quantitative value such as EF. Further, according to the image processing apparatus 10 in the first embodiment, when the quantitative value such as EF is acquired by manual input of the operator, contour candidates corresponding to the quantitative value can be presented to the operator, and the operator can select desired contour data out of the candidates. As a result, it is possible to acquire contour data that is desirable and also corresponding to the quantitative value such as EF.

### Second Embodiment

The image processing apparatus according to the second embodiment is provided separately from the ultrasonic diagnostic apparatus as the medical diagnostic imaging apparatus.

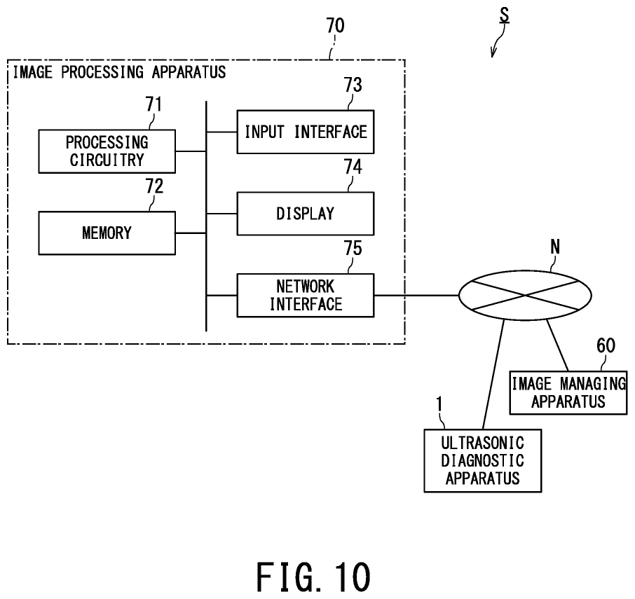

FIG. 10 is a schematic view showing an example of a configuration of a medical image system including the image processing apparatus according to the second embodiment.

FIG. 10 shows a medical image system S including an ultrasonic diagnostic apparatus 1 as the medical diagnostic imaging apparatus. The medical image system S includes the above-mentioned ultrasonic diagnostic apparatus 1 and the image processing apparatus 70 as an image display apparatus. The image processing apparatus 70 is a workstation that performs various image processing on image data, a portable data processing terminal such as a tablet terminal, or the like. The image processing apparatus 70 is connected to the ultrasonic diagnostic apparatus 1 via the network N to be capable of communication.

The image processing apparatus 70 includes processing circuitry 71, a memory 72, an input interface 73, a display 74, and a network interface 75. The processing circuitry 71, the memory 72, the input interface 73, the display 74, and the network interface 75 are assumed to have the same configurations as the processing circuitry 17, the main memory 18, the input interface 19, the display 40, and the network interface 16 shown in FIG. 1, thereby the description will be omitted.

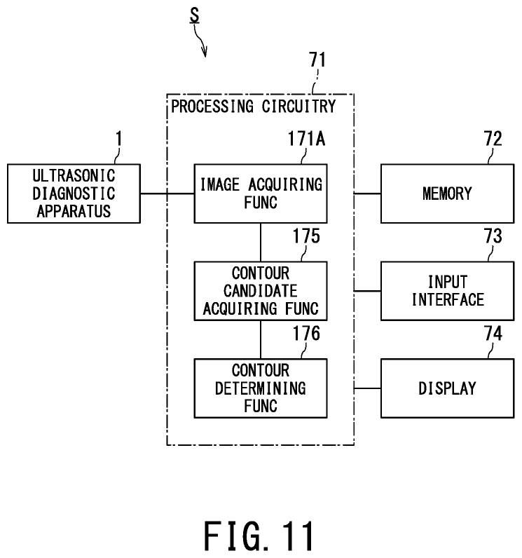

FIG. 11 is a block diagram showing an example of functions of the image processing apparatus 70.

The processing circuitry 71 readouts and executes a computer program stored in the memory 72 or directly embedded in the processing circuitry 71, so as to realize the image acquiring function 171A, the contour candidate acquiring function 175, and the contour determining function 176. Hereinafter, the case where the functions 171A, 175 and 176 are realized by a computer program will be described as an example, but the present invention is not limited to this case. All or part of the functions 171A, 175 and 176 may be provided in the image processing apparatus 70 as a function of a circuit such as an ASIC.

In FIG. 11, the same members as those shown in FIG. 2 are designated by the same reference numerals, and the description thereof will be omitted.

The image acquiring function 171A includes a function of acquiring ultrasonic image data stored by the storing control function 172 of the ultrasonic diagnostic apparatus 1. Specifically, the image acquiring function 171A controls the network interface 75 to acquire moving image data, which are stored by the storing control function 172 of the ultrasonic diagnostic apparatus 1, from the ultrasonic diagnostic apparatus 1 or the image managing apparatus 60 via the network N. The image acquiring function 171A is an example of an image acquiring unit.

Subsequently, an operation of the image processing apparatus 70 will be described.

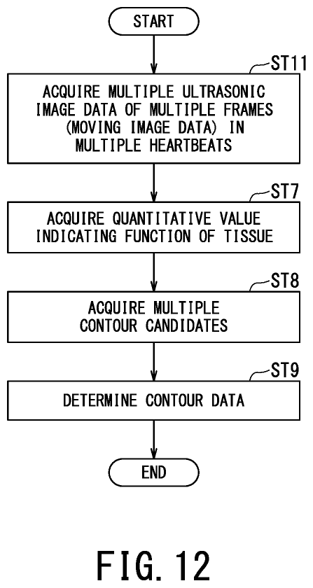

FIG. 12 is a flowchart showing an example of the operation of the image processing apparatus 70. In FIG. 12, the reference numeral “ST” with a number indicates each step of the operation. In FIG. 12, the same steps as those in FIG. 5 are designated by the same reference numerals, and the description thereof will be omitted.

The image acquiring function 171A of the image processing apparatus 70 controls the network interface 75 to acquire moving image data, which are stored by the storing control function 172 of the ultrasonic diagnostic apparatus 1, from the ultrasonic diagnostic apparatus 1 or the image managing apparatus 60 via the network N (step ST11).

According to the image processing apparatus 70 in the second embodiment, when the quantitative value such as EF is acquired by using the “Auto EF” function, contour candidates corresponding to the quantitative value can be presented to the operator, and the operator can select desired contour data out of the candidates. As a result, it is possible to acquire contour data that is desirable and also corresponding to the quantitative value such as EF. Further, according to the image processing apparatus 70 in the second embodiment, when the quantitative value such as EF is acquired by manual input of the operator, contour candidates corresponding to the quantitative value can be presented to the operator, and the operator can select desired contour data out of the candidates. As a result, it is possible to acquire contour data that is desirable and also corresponding to the quantitative value such as EF.

According to at least one embodiment described above, it is possible to present to the operator the multiple contours corresponding to the quantitative value indicating the function of the tissue.

While certain embodiments have been described, these embodiments have been presented by way of example only, and are not intended to limit the scope of the inventions. Indeed, the novel methods and systems described herein may be embodied in a variety of other forms; furthermore, various omissions, substitutions, changes, and combinations of embodiments in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to
acquire ultrasonic image data by ultrasonic scanning,
acquire multiple contour candidates based on a quantitative value indicating a function of a tissue, and superimpose the multiple contour candidates on the acquired ultrasonic image data to display the multiple contour candidates on a display, and
determine contour data out of the multiple contour candidates.

2. The ultrasonic diagnostic apparatus according to claim 1, wherein
the processing circuitry is configured to acquire the multiple contour candidates of a left ventricular endocardium as the tissue based on the quantitative value manually input via an input interface.

3. The ultrasonic diagnostic apparatus according to claim 2, wherein
the processing circuitry is configured to acquire the multiple contour candidates of the left ventricular endocardium according to an adjustment of the quantitative value.

4. The ultrasonic diagnostic apparatus according to claim 1, wherein
the processing circuitry is configured to preset the number of the multiple contour candidates.

5. The ultrasonic diagnostic apparatus according to claim 4, wherein
the processing circuitry is configured to
preset a feature point, and
acquire the multiple contour candidates passing the feature point.

6. The ultrasonic diagnostic apparatus according to claim 5, wherein
the feature point is at least one of an apex and an annulus of a heart.

7. The ultrasonic diagnostic apparatus according to claim 6, wherein
The processing circuitry is configured to, when the preset number of the multiple contour candidates cannot be acquired due to the limitation of the quantitative value and the feature point, (A) display “warning” sign on a display screen, (B) recommend changing the quantitative value via a display screen, (C) recommend changing the number of the multiple contour candidates via a display screen, or (D) recommend changing the feature point via a display screen.

8. An image processing apparatus comprising: processing circuitry configured to
acquire ultrasonic image data,
acquire multiple contour candidates based on a quantitative value indicating a function of a tissue, and superimpose the multiple contour candidates on the acquired ultrasonic image data to display the multiple contour candidates on a display, and
determine contour data out of the multiple contour candidates.

9. An image processing method comprising steps of
acquiring ultrasonic image data,
acquiring multiple contour candidates based on a quantitative value indicating a function of a tissue, and superimpose the multiple contour candidates on the acquired ultrasonic image data to display the multiple contour candidates on a display, and
determining contour data out of the multiple contour candidates.
